(19) United States
(12) Patent Application Publication
(10) Pub. No.: US 2020/0066421 A1
(43) Pub. Date: Feb. 27, 2020

(54) Wrapping-Tape Insulating System for Electrical Machines, Use Therefor, and Electrical Machine

(71) Applicant: Siemens Aktiengesellschaft, München (DE)

(72) Inventors: Mario Brockschmidt, Essen (DE); Jürgen Huber, Erlangen (DE); Regina Mühlberg, Halle (DE); Irene Olbrich, Berlin (DE); Torsten Rossow, Berlin (DE); Matthias Übler, Ursensollen (DE); Dieter Schirm, Breitengüßbach (DE)

(73) Assignee: Siemens Aktiengesellschaft, München (DE)

(21) Appl. No.: 16/482,198

(22) PCT Filed: Jan. 25, 2018
(86) PCT No.: PCT/EP2018/051814

§ 371 Date: Jul. 30, 2019

(30) Foreign Application Priority Data
Jan. 31, 2017 (DE) 10 2017 201 498.9

(51) Int. Cl. H01B 3/40; H02K 3/30; H02K 15/12; C08L 63/00; C07D 233/88; H01B 3/04

(52) U.S. Cl. CPC H01B 3/40; H02K 3/30; H01B 3/04; C08L 63/00; C07D 233/88; H02K 15/12

(57) ABSTRACT

A wrapping tape insulation system is provided which includes an insulating tape preferably produced by vacuum pressure impregnation. The provided tape comprises a tape adhesive, a tape accelerator and a resin wherein the tape accelerator may comprise an imidazole covalently bonded via the amino function to an acrylate by aza-Michael addition.

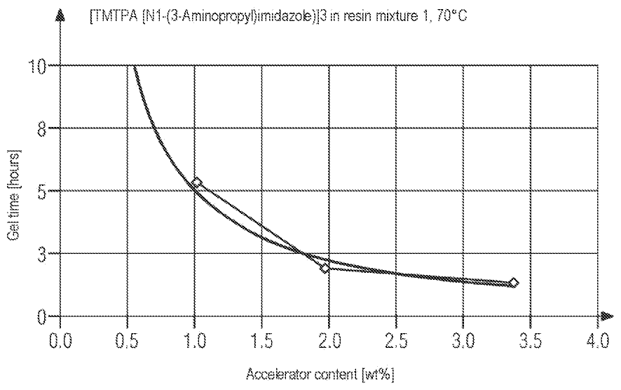

### CROSS-REFERENCE TO RELATED APPLICATIONS

This application is a U.S. National Stage Application of International Application No. PCT/EP2018/051814 filed Jan. 25, 2018, which designates the United States of America, and claims priority to DE Application No. 10 2017 201 498.9 filed Jan. 31, 2017, the contents of which are hereby incorporated by reference in their entirety.

### FIELD OF THE INVENTION

The present invention relates to wrapping-tape insulating systems for electrical machines, use thereof, and electrical machines.

In addition, the invention relates to a wrapping-tape insulating systems for electrical machines, and more particularly to systems having a tape accelerator that is more vacuum-stable than the prior art.

### BACKGROUND OF THE INVENTION

A wrapping-tape insulating system is used for example for the insulation of stator coils in electrical machines. More particularly, insulating tape is used to wrap around a conductor or a bundle of conductors of electrical machines.

A wrapping-tape insulating system for the medium-voltage and high-voltage sectors fundamentally comprises an insulating tape, of which the winding consists. This winding is impregnated in a special vacuum impregnation process (vacuum pressure impregnation, VPI) using a thermally curable epoxy resin to produce the completed wrapping-tape insulating system.

The insulating tape comprises a sheetlike, breakdown-resistant inorganic material, such as mica platelets and/or fine mica layers, which is applied on a flexible backing such as foil or glass fabric, and which is joined to the backing and to one another and, optionally, to a concluding outer ply and/or a further ply, by means of a tape adhesive.

This tape adhesive comprises a tape accelerator ultrafinely divided and/or dissolved therein. The purpose of the tape accelerator is to gel a highly mobile impregnating resin which is applied to the windings in the vacuum pressure impregnation (VPI). After the gelling at elevated temperature, the impregnated stator windings are cured thermally, for example, in what is called the laminated core of the stator.

To allow the insulating tapes to be stored for a long time prior to impregnation, in the conventional insulating tape the binder-accelerator mixture is selected such that this mixture undergoes virtually no curing at room temperature.

One such insulating tape is known from DE 38 24 254 A1. EP 0424376B1 discloses corresponding tape adhesives and tape accelerators which exhibit sufficient storage stability.

The tape adhesives disclosed therein are the 1:4-molar adducts of bisphenols, more particularly of bisphenol A, and cycloaliphatic epoxy resins, more particularly 3,4-epoxycyclohexylmethyl 3,4-epoxycyclohexanecarboxylate.

This binder, in its theoretical molecular structure after quantitative conversion, presumably contains almost exclusively cycloaliphatic oxirane functionalities as well as the hydroxyl groups likewise created through the addition reaction. It is further disclosed in EP 0424376B1 that the associated tape accelerator is preferably a 1:3-molar adduct of trimethylolpropane triacrylate and N-ethylpiperazine.

The tape accelerator and/or the tape adhesive in the mica tape assembly are more particularly selected chemically such that there is no premature and unwanted curing in the course of storage at room temperature. This ensures the processability of the mica tape. Following impregnation of the stator coils containing tape adhesive, the gelling of the glycidyl ether-based epoxy resin takes place very rapidly, since terminal oxirane functionalities in interaction with acyl anhydrides are subject to very rapid polymerization. As a result of this chemically attuned interaction, it is possible ultimately to realize the desired storage stability of the unimpregnated mica tape and also the rapid gelling of the impregnated stator.

On account of toxicity concerns against the unrestricted use of phthalic anhydride, VPI resins based on epoxide that are used in the future will be free of phthalic anhydride or completely free of anhydride. This is expressed more particularly in the Echa Europe list.

The new curing catalysts will be attuned to the anhydride-free impregnating compositions. There will be increased use of anhydride-free impregnating compositions, as known from the earlier patent applications: DE 102014219844.5; DE 102014221715.6; DE 102015205328.8, DE 102015202053.3; DE 102015208527.9; and DE 102015204885.3, the disclosure content of which is hereby made part of the present description.

From DE 10 2015 214 872 A1 it is already known that nitrogen heterocycles, such as imidazoles, for instance, especially imidazoles with alkyl substitution at positions 1 and 2, deliver effective tape accelerators for phthalic anhydride-free epoxy resins based on bisphenol A and/or bisphenol F diglycidyl ether.

Thus, for example, a phthalic anhydride-free and also binder-free bisphenol F diglycidyl ether which is gelled with N-ethylpiperazine derivative of trimethylolpropane triacrylate and is subjected to anionically polymerizing curing at 145° C. for ten hours produces only a glass transition of around 90° C., whereas the phthalic anhydride-containing mixture with binder and tape accelerator, upon identical curing, develops a glass transition of around 160° C.

If, in contrast, alkylimidazole, more particularly 1,2-disubstituted diimidazoles, is used instead of the N-ethyl-piperazine derivative of trimethylolpropane triacrylate, then the glass transition temperature rises to above 130° C. Consequently, in the new generation of tape accelerators attuned to anhydride-free epoxy resin mixtures, preferably imidazoles will be used, more particularly alkylimidazoles, as tape accelerators.

A disadvantage of the alkylimidazoles known for this use, for example, from DE 10 2015 214 872 A1 and/or from DE 102015213534 A1 and DE 10 2015204885 A1, however, is that it has emerged that the vapor pressures of the alkylimidazoles are relatively high, resulting in partial expulsion of the imidazoles used from the mica tape during the elevated-temperature evacuation phases that are a standard component of the vacuum impregnation process. This expulsion is very disadvantageous not least because at elevated temperature, during the evacuation phase and preliminary drying phase of the windings to be impregnated, they can migrate from the tape adhesive, and may lead to accumulation of the volatile alkylimidazole at relatively cold locations and/or to contamination of the impregnating resin.

### SUMMARY OF THE INVENTION

It is an object of the present invention, therefore, to provide an insulating tape and in particular an alkylimidazole tape accelerator for an insulating tape that can be used together with anhydride-free VPI resins. A further object of the invention is to specify an insulating system, a coil, and an electrical machine having an insulating tape impregnated with an anhydride-free resin of this kind.

The achievement of the object is provided by the subject matter of the invention, as disclosed in the description and the claims.

A subject of the present invention, accordingly, is a wrapping-tape insulating system comprising an insulating tape with at least one tape adhesive, tape accelerator ultrafinely divided therein, and an anhydride-free impregnating resin, wherein there is at least one tape accelerator based on an amino-imidazole and/or aminoalkyl-imidazole and/or any desired derivatives thereof, bonded via the amino function to an acrylate.

A further subject of the invention is the use of such a wrapping-tape insulating system in electrical machines, preferably in rotating electrical machines, more preferably in rotating electrical machines in the medium-voltage and high-voltage range, and also in electrical switchgear, medium-voltage and high-voltage applications, bushings, transformer bushings, generator bushings and/or HVDC bushings, and also in corresponding semi-finished products.

Lastly, a further subject of the invention are electrical machines, preferably rotating electrical machines, more preferably rotating electrical machines in the medium-voltage and high-voltage range, and also electrical switchgear, medium-voltage and high-voltage applications, bushings, transformer bushings, generator bushings and/or HVDC bushings, and also corresponding semi-finished products, which comprise a wrapping-tape insulating system of this kind.

### BRIEF DESCRIPTION OF THE DRAWING

The single FIGURE shows a graph which represents the gel time—measurement as per Iso 9396 at 70° C. The formulation involved is the new tape accelerator class of the aza-Michael adduct of TMPTA and 3-aminopropyl-1H-imidazole in epoxy resin mixture 1.

### DETAILED DESCRIPTION

A general realization of the invention is that the 1H-imidazole derivatives already described in DE 102015 214872, an example being 1H-2-alkylimidazole, can indeed be vacuum-stabilized by addition onto a CC double bond of acrylic esters, but that the steric hindrance of the side chain in position 2 of the imidazole ring hinders the addition onto the CC double bond and means that the tape accelerator always still contains 1H-2-alkylimidazole which is still “free”, i.e., is not bonded to the acrylate and is therefore present with high vapor pressure at already low temperatures, this imidazole being expelled by way of the vacuum impregnation.

Through the reaction with amino-imidazole by way of the amino function, by means of the aza-Michael addition, for example, onto the same acrylates, it is possible to obtain tape accelerators which exhibit a high reactivity toward the impregnating resin which penetrates during the vacuum impregnation process, while at the same time having a low reactivity toward the tape adhesive. The critical factor here is an extremely low vapor pressure in the temperature range up to 80° C., so that under the conditions of the vacuum pressure impregnation, there can be no risk of the thermosetting, anhydride-free epoxy resin mixture becoming infected by vapors of accelerating and activating constituents from the tape adhesive-accelerator mixture, as may occur with the existing accelerator substances known from DE 10 2015 214 872—the disclosure content of which is hereby made part of the present description.

According to one advantageous embodiment of the invention, the tape adhesive-accelerator mixture is formulated so that under the conditions of the vacuum impregnation, they are consumed by reaction with an anhydride-free impregnating composition with gelling times of 1 hour to 15 hours at impregnating temperature.

According to one embodiment of the invention there is at least one tape accelerator which is an amino-imidazole bonded covalently to a higher acrylate via the amino function.

As amino-imidazole it is possible for example to use aminoalkyl-imidazole, preferably 1-(aminoalkyl)imidazole, 1H-2-aminoimidazole, 1H-2-(aminoalkyl)imidazole, 1H-4-aminoimidazole, 1H-4-(aminoalkyl)imidazole, 1H-5-aminoimidazole, 1H-5-(amino-alkyl)imidazole, and also the corresponding derivatives. Examples of suitable derivatives are: 1-(3-aminopropyl)imidazole (CAS No. 5036-48-6), 1H-2-aminoimidazoles (CAS No. 7720-39-0), 1H-2-aminomethylimidazole 1H-2-aminoethylimidazole 1H-2-aminopropylimidazole 1H-2-aminobenzimidazoles (CAS No. 934-32-7).

Further examples are imidazoles which can be derived from the following structures:

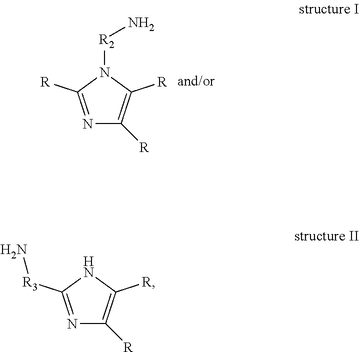

where

R₂═(—CH₂—)ₙ wherein n=1, 2, 3, . . . , 10,

R₃═(—CH₂—)ₙ wherein n=1, 2, 3, . . . , 10,

and R (identical and/or nonidentical)=methyl, ethyl, n-propyl, isopropyl, n-butyl, isobutyl, tert-butyl, aryl, more particularly phenyl.

They are then bonded—in particular covalently—to a higher acrylate via an aza-Michael addition, for example.

As higher acrylate it is possible for example to use diacrylates, triacrylates, tetra-, penta-, and/or hexaacrylates, more particularly

trimethylolpropane triacrylate (TMPTA, CAS No. 15625-89-5), trimethylolpropane propoxylate triacrylate (no synonym, CAS No. 53879-54-2),

pentaerythritol tetraacrylate (PETA, CAS No. 4986-89-4),

dipentaerythritol pentaacrylate/dipentaerythritol hexaacrylate (CAS No. 60506-81-2).

The desired high glass transition temperatures in the materials system with the phthalic acid-free, epoxy resin-based impregnating resin are achieved as a result of the addition onto the higher acrylates (aza-Michael addition); cf. Table 1.

TABLE 1
Glass transition ranges of the new aza-Michael adducts

| | Glass transition range [° C.] | | | |
|---|---|---|---|---|
| | per DSC run (half height), 10 K/min, in | | | |
| | Epoxy resin | | Epoxy resin | |
| | mixture 1 | | mixture 2 | |
| TMPTA (N1-3-Amino- | 10 h/ | 10 h/ | 10 h/ | 10 h/ |
| propylimidazole)₃ | 145 C.^{[}*^{]} | 160 C.^{[}*^{]} | 145 C.^{[}*^{]} | 160 C.^{[}*^{]} |
| 0.5 wt % | not gelled | not gelled | not gelled | not gelled |
| 1.0 wt % | 52 | 55 | 51 | 55 |
| 2.0 wt % | 140 | 146 | 142 | 135 |
| 3.0 wt % | 139 | 135 | 143 | 131 |

^{[}*^{]}curing conditions

With the new types of tape accelerator disclosed here for the first time, from just two wt % fractions, tape accelerators are formed which have productive glass transition ranges in the cured state of more than 140° C., whereas with the tape accelerators from the prior art, glass transitions at this level result only at or above a tape accelerator content of more than three wt %. This is later reflected advantageously in a lower amount of tape accelerator applied per unit area.

The innovative tape accelerators also prove advantageous in terms of the gelling tendency of the impregnating resin used. Hence the gel time is reduced by around 40-50% relative to the prior art, as determined by relevant methods known to the skilled person, e.g., ISO 9396.

The epoxy resin basis of the impregnating resin may for example be one or more compounds selected from the group recited below:
diglycidyl ether derivative of a bisphenol X, wherein
   X=A, CAS No. 80-05-7,
   X═B, CAS No. 77-40-7,
   X=E, CAS No. 2081-08-5,
   X═F, CAS No. 620-92-8,
   X=AP, CAS No. 1571-75-1
glycidyl ester derivatives, i.e., diglycidyl esters of aliphatic, cycloaliphatic and/or aromatic dicarboxylic acids,
glycidyl ether derivatives of phenolic novolacs (so-called “epoxy phenol novolacs”)
glycidyl ether derivatives of cresolic novolacs (so-called “epoxy cresol novolac”)

and also any desired mixtures of the aforesaid compounds.

The graph shown in the single figure represents the gel time—measurement as per Iso 9396 at 70° C. The formulation involved is the new tape accelerator class of the aza-Michael adduct of TMPTA and 3-aminopropyl-1H-imidazole in epoxy resin mixture 1.

For particularly simple chemical synthesis of the tape accelerators presented here for the first time, and bonded to the acrylates via the amino function, use is made in particular, as reactants, of acrylates which are liquid at room temperature with imidazoles which melt at relatively low temperatures.

The synthesis may alternatively take place in a suitable inert solvent, as known to the skilled person.

The stoichiometry of the reaction is a function—as familiar to the relevant skilled person—of the functionality of the acrylate.

Suitable tape adhesives are preferably tape adhesives free from oxirane groups, since otherwise the storage stability with the tape accelerators in accordance with the invention is questionable. Preferred for use as tape adhesives are copolyesters, diols and/or higher alcohols, and also any desired mixtures thereof. Especially suitable in this context are the thermoplastic compounds. Also suitable as tape adhesives are linear compounds free from oxirane groups.

Examples of suitable tape adhesives are:

tricyclomethanedimethanol (CAS No. 26896-48-0 or 26160-83-8),

trimethylolpropane (CAS No. 77-99-6),

dendritic, hydroxy-functional polymers (CAS No. 326794-48-3 or 462113-22-0),

polycaprolactone triols (CAS No. 37625-56-2),

polycaprolactone tetrols (CAS No. 35484-93-6)

and also the oxirane group-free tape adhesive materials described in WO 2016/150764.

Below is the structure III, an exemplary embodiment of the tape accelerator which shows adducts of amino-imidazole onto trimethylolpropane triacrylate (TMPTA):

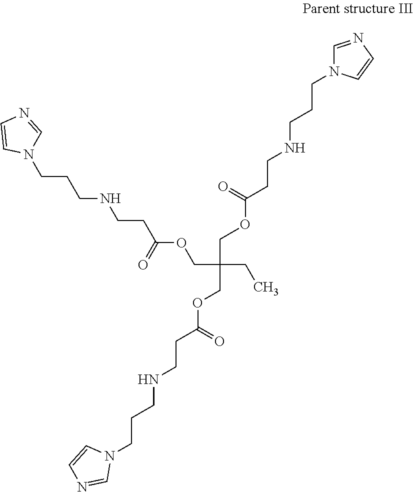 [SMILES: CCC(COC(=O)CCNCCCn1ccnc1)(COC(=O)CCNCCCn1ccnc1)COC(=O)CCNCCCn1ccnc1]

Shown below are further parent structures IV to VI, which represent exemplary embodiments of a tape accelerator suitable for the purposes of the invention. In principle the structure is always the adduct of TMPTA and one or more amino-1H-imidazole derivative(s).

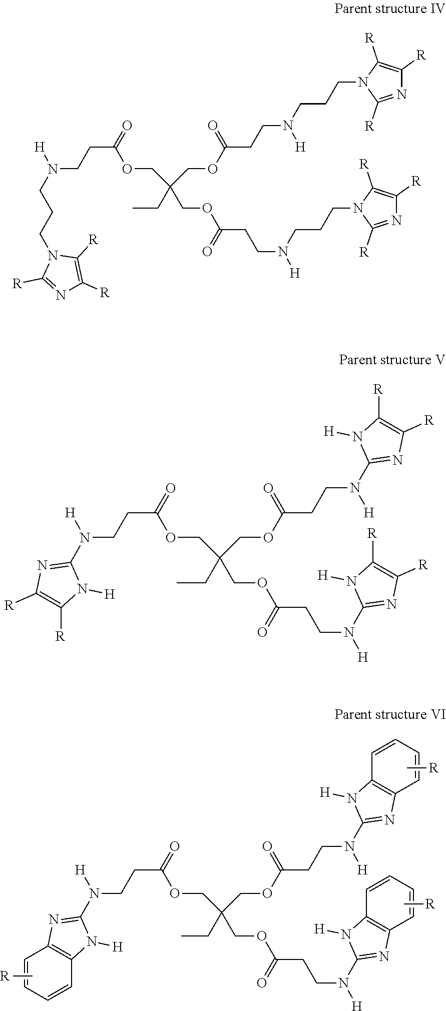

For the structural formulae IV to VI shown here:

R=identical and/or nonidentical and ═H, methyl, ethyl, n-propyl, isopropyl, n-butyl, isobutyl, tert-butyl, phenyl and/or mono-, di-, tri-, tetra-, pentasubstituted phenyl, wherein the substituents on the phenyl radical may in turn be identical or nonidentical and may have been selected from the following group:

R_{phenyl}=alkyl (linear and branched), alkoxy, —F, —Cl, —Br, —I, aldehyde, ketone, acyl ester, acyl amide, phosphonic acid derivative and/or sulfonic acid derivative.

Further embodiment examples of adducts of amino-1H-imidazoles and trimethylolpropane propoxylate triacrylate which are suitable as tape accelerators in accordance with the invention are shown in the structures VII to IX below:

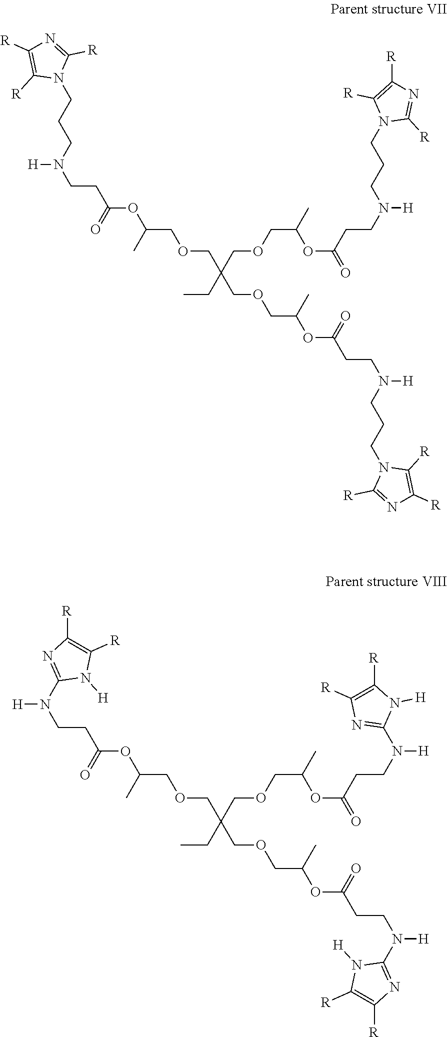

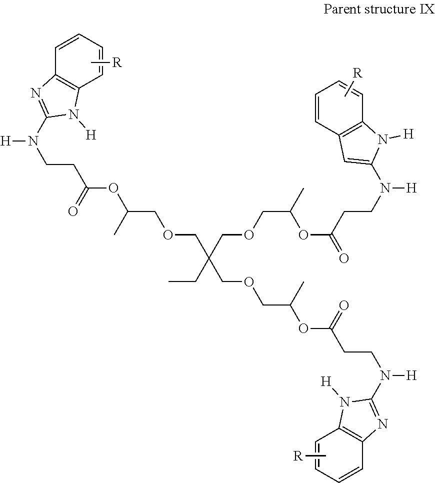

For the structural formulae VII to IX shown here:

R=identical and/or nonidentical and ═H, methyl, ethyl, n-propyl, isopropyl, n-butyl, isobutyl, tert-butyl, phenyl and/or mono-, di-, tri-, tetra-, pentasubstituted phenyl, wherein the substituents on the phenyl radical may in turn be identical or nonidentical and may have been selected from the following group:

R_{phenyl}=alkyl (linear and branched), alkoxy, —F, —Cl, —Br, —I, aldehyde, ketone, acyl ester, acyl amide, phosphonic acid derivative and/or sulfonic acid derivative.

Further embodiment examples of adducts of amino-1H-imidazoles and pentaerythritol tetraacrylate (PETA), which are suitable as tape accelerators in accordance with the invention, are shown in the structures X to XII below:

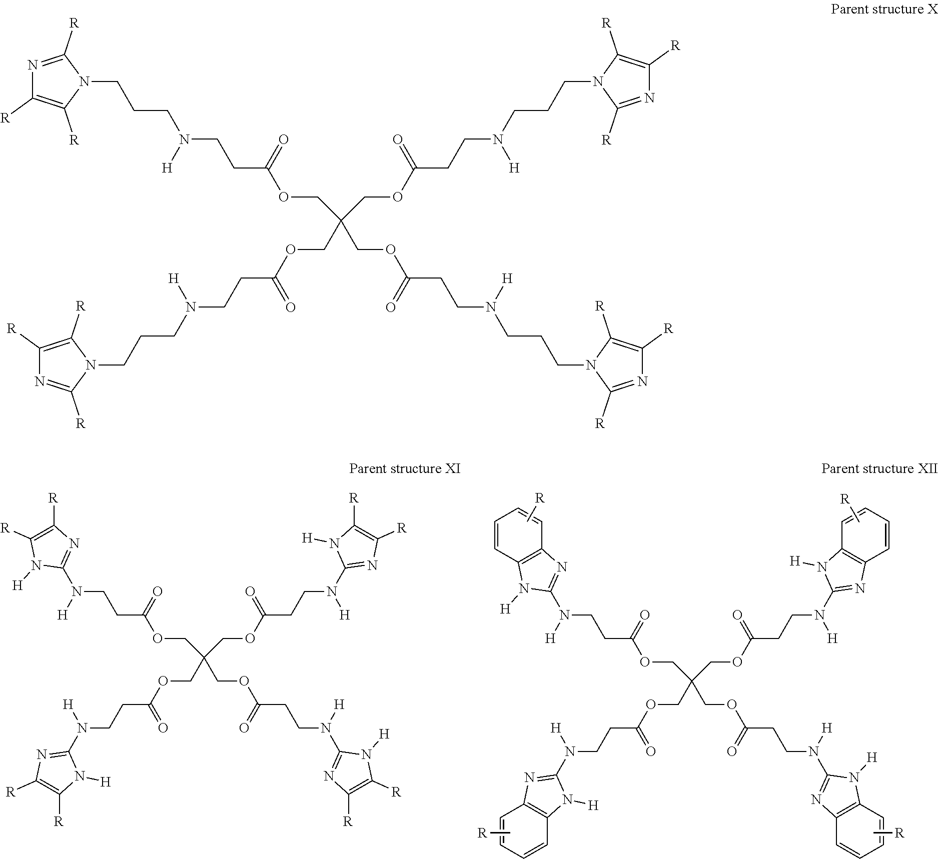

The parent structures X to XII show adducts of amino-imidazoles onto pentaerythritol tetraacrylate (PETA).

For the structural formulae X to XII shown:

R identical and/or nonidentical and selected from the following group:

R═H, methyl, ethyl, n-propyl, isopropyl, n-butyl, isobutyl, tert-butyl, phenyl and/or mono-, di-, tri-, tetra-, pentasubstituted phenyl,

wherein the substituents on the phenyl radical may in turn be identical or nonidentical and may have been selected from the following group:

R_{phenyl}=alkyl (linear and branched), alkoxy, —F, —Cl, —Br, —I, aldehyde, ketone, acyl ester, acyl amide, phosphonic acid derivative and/or sulfonic acid derivative.

Further embodiment examples of adducts of amino-1H-imidazoles and dipentaerythritol pentaacrylate and/or hexaacrylate (DPHA), which are suitable as tape accelerators in accordance with the invention, are shown in the following structure XIII with possible radicals as depicted in structures XIV to XVI:

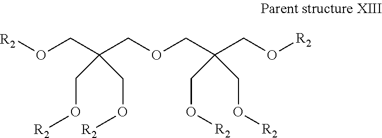

wherein

R₂═H and/or

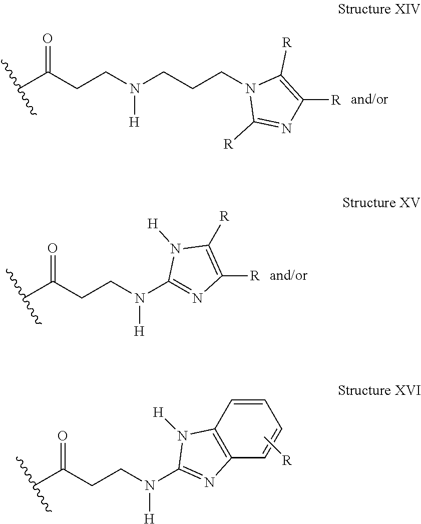

For the structural formulae XIII to XVI shown:

R=identical and/or nonidentical and

R═H, methyl, ethyl, n-propyl, isopropyl, n-butyl, isobutyl, tert-butyl, phenyl and/or mono-, di-, tri-, tetra-, pentasubstituted phenyl,

wherein the substituents on the phenyl radical may in turn be identical or nonidentical and may have been selected from the following group:

R_{phenyl}=alkyl (linear and branched), alkoxy, F, Cl, Br, I, aldehyde, ketone, acyl ester, acyl amide, phosphonic acid derivative and/or sulfonic acid derivative

R₂ as indicated in the structures XIV to XVI.

In comparison with the prior art as known from EP 0424376 B1, in other words, for example, the 1:3 molar adduct of trimethylol-propane triacrylate (“TMPTA”) and N-ethylpiperazine, the adducts described here, of triacrylates, but more particularly also of the tetra- and penta-/hexaacrylates, with 1-(aminoalkyl)imidazoles, 1H-2-aminoimidazoles, 1H-2-(aminoalkyl)imidazoles, are compounds of particular interest as tape accelerators, because
a) the glass transition temperatures achievable by anionic polymerization with phthalic anhydride-free, diglycidyl ether-based impregnating resins are much higher than when using the N-methylpiperazine and N-ethylpiperazine adducts of TMPTA
b) 1H-2-alkylimidazoles and (1,2-alkyl)imidazoles gel and cure phthalic anhydride-free impregnating resins with lower amounts than an N-alkylpiperazine-TMPTA adduct (alkyl=for example methyl, ethyl)

Through the covalent attachment of the 1-(aminoalkyl)imidazoles, 1H-2-aminoimidazoles, 1H-2-(aminoalkyl)imidazoles, via the amino group located laterally relative to the imidazole ring, onto the TMPTA used to date, via an aza-Michael coupling, it is now possible to substitute the N-ethylpiperazine derivative of TMPTA, unsuitable for phthalic anhydride-free epoxy resins, with an imidazole-TMPTA variant which is suitable for the new generation of impregnating resins without acyl anhydrides.

Of particular importance here are 1H-2-aminoimidazoles and 1H-2-(aminoalkyl)imidazoles, since even after addition onto TMPTA, for example, they still have the 1H-imidazole function, which is available for later curing of the phthalic anhydride-free epoxy resin.

In numerous preliminary experiments it has emerged that, for example, 1,2-dimethylimidazole at 2 wt %, based on the phthalic anhydride-free impregnating resin, more particularly an epoxy resin, furnishes high glass transitions of up to 140° C. under otherwise identical curing scenarios, whereas the piperazine-based tape accelerator produces only around 90° C. as a glass transition in phthalic anhydride-free, glycidyl ether epoxy resins.

By virtue of the high vapor pressure of the 1H-2-alkylimidazoles and of the (1,2-alkyl)imidazoles, and of the high fluidity, the dispersing of pure imidazoles into the mica tape binder is in fact associated with the later risk that the evacuation phase at around 50° C. to 80° C., under subatmospheric pressure, leads to evaporation or migration of the volatile alkylimidazole, which accumulates at relatively cold locations. During the resin flooding phase, accordingly, resin contamination is likely. Through covalent attachment of the alkylimidazoles onto an acrylate, there is a drastic increase in viscosity as a result of the construction of an addition tape accelerator molecule having a dynamic viscosity of up to 8 Pa*s or more at room temperature. Consequently there is effective retardation of migration from the mica tape binder.

The tape accelerators disclosed here for the first time are preferably vacuum stable, which means, for example, that they have a vapor pressure of less than 10⁻³, more particularly below 10⁻⁴, at a temperature of 50° C. to 80° C.

In accordance with the invention, imidazoles connected to acrylates via an amino function are presented here for the first time as tape accelerators or curing catalysts in wrapping-tape insulating systems. These innovative tape accelerators are inert toward the proposed tape adhesives free from oxirane groups, and exhibit a curing-catalytic effect relative to the epoxy-based impregnating resins in a vacuum pressure impregnation.

What is claimed is:

1. A wrapping-tape insulating system produced by vacuum pressure impregnation, the system comprising
an insulating tape with at least one tape adhesive,
a tape accelerator mixed in the tape adhesive, and
an anhydride-free impregnating resin,
wherein the tape accelerator comprises an amino-imidazole, amino-imidazole derivatives and mixtures thereof, bonded via the amino function to an acrylate by aza-Michael addition.

2. The wrapping-tape insulating system as claimed in claim 1, wherein the amino-imidazole is selected from the group of imidazoles consisting of aminoalkyl-imidazoles, 1-(aminoalkyl)imidazole, 1H-2-aminoimidazole, 1H-2-(aminoalkyl)imidazole, 1H-4-aminoimidazole, 1H-4-(amino-alkyl)imidazole, 1H-5-aminoimidazole, 1H-5-(aminoalkyl)-imidazole, 1-(3-amino-propyl)imidazole,
1H-2-aminoimidazoles,
1H-2-aminomethylimidazole,
1H-2-aminoethylimidazole,
1H-2-aminopropylimidazole, and
1H-2-aminobenzimidazoles.

3. The wrapping-tape insulating system as claimed in claim 1, wherein the amino-imidazole is selected from the group of imidazoles derivable from the following structures I and II: 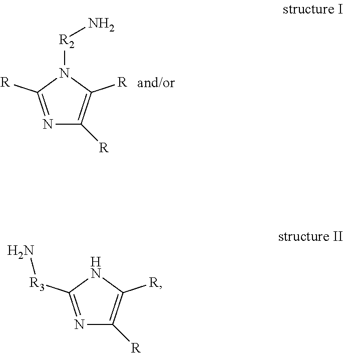
where
R₂═(—CH₂—)ₙ wherein n=1, 2, 3, . . . , 10,
R₃═(—CH₂—)ₙ wherein n=1, 2, 3, . . . , 10,
and R (identical and/or nonidentical)=methyl, ethyl, n-propyl, isopropyl, n-butyl, isobutyl, tert-butyl, aryl, more particularly phenyl.

4. The wrapping-tape insulating system as claimed in claim 1, wherein the -acrylate is selected from the group of acrylates consisting of diacrylates, triacrylates, tetra-acrylates, penta-acrylates, hexa-acrylates and mixtures thereof.

5. The wrapping-tape insulating system as claimed in claim 1, wherein the acrylate is selected from the group of acrylates consisting of
trimethylolpropane triacrylate,
trimethylolpropane propoxylate triacrylate,
pentaerythritol tetraacrylate,
dipentaerythritol pentaacrylate/dipentaerythritol hexaacrylate and mixtures therof.

6. The wrapping-tape insulating system as claimed in claim 1, wherein the the tape adhesive comprises a copolyester or a diol and mixtures thereof.

7. The wrapping-tape insulating system as claimed in claim 1, wherein the tape accelerator is present in the insulating tape in an amount of less than about 10 wt %, based on the amount of impregnating resin.

8. The wrapping-tape insulating system as claimed in claim 1, wherein the tape adhesive is present in the insulating tape in an amount in the range from about 1 to about 30 wt %, based on the amount of impregnating resin.

9. The wrapping-tape insulating system as claimed in claim 1, wherein the tape accelerator is in the form of a compound having the following structure III:

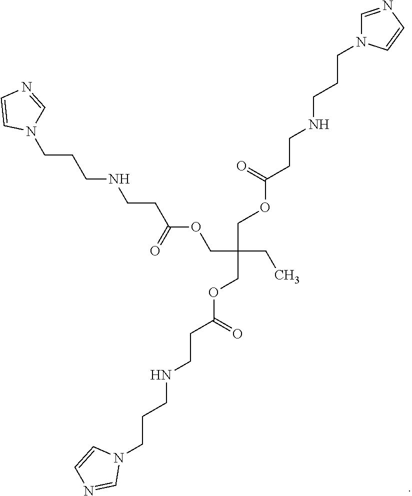 [SMILES: CCC(COC(=O)CCNCCCn1ccnc1)(COC(=O)CCNCCCn1ccnc1)COC(=O)CCNCCCn1ccnc1]

10. The wrapping-tape insulating system as claimed in claim 1, wherein the tape accelerator is in the form of a compound whose structure is derived from at least one of the following parent structures IV, V and VI: 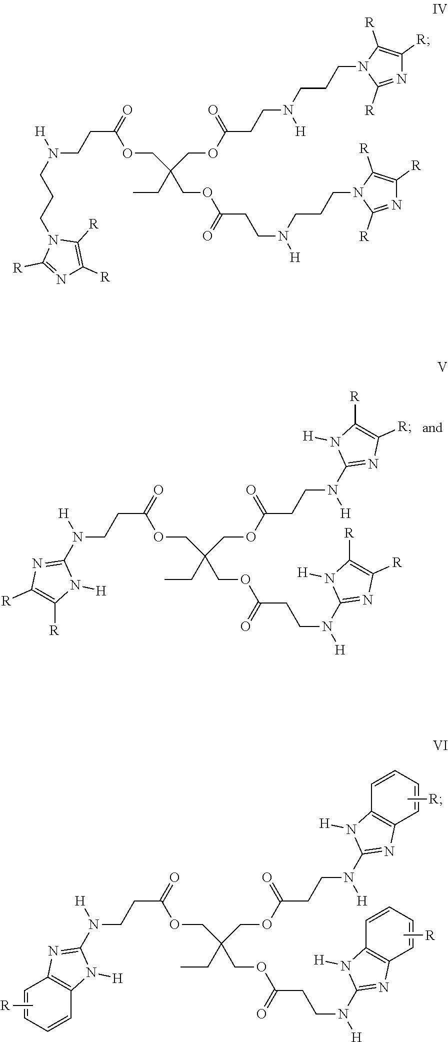
wherein
R=identical and/or nonidentical and ═H, methyl, ethyl, n-propyl, isopropyl, n-butyl, isobutyl, tert-butyl, phenyl and/or mono-, di-, tri-, tetra-, pentasubstituted phenyl, wherein the substituents on the phenyl radical may in turn be identical or nonidentical and may have been selected from the following group:
R_{phenyl}=alkyl (linear and branched), alkoxy, —F, —Cl, —Br, —I, aldehyde, ketone, acyl ester, acyl amide, phosphonic acid derivative and/or sulfonic acid derivative.

11. The wrapping-tape insulating system as claimed in claim 1, wherein the tape accelerator is in the form of a compound whose structure is derived from at least one of the following parent structures VII, VIII and IX: 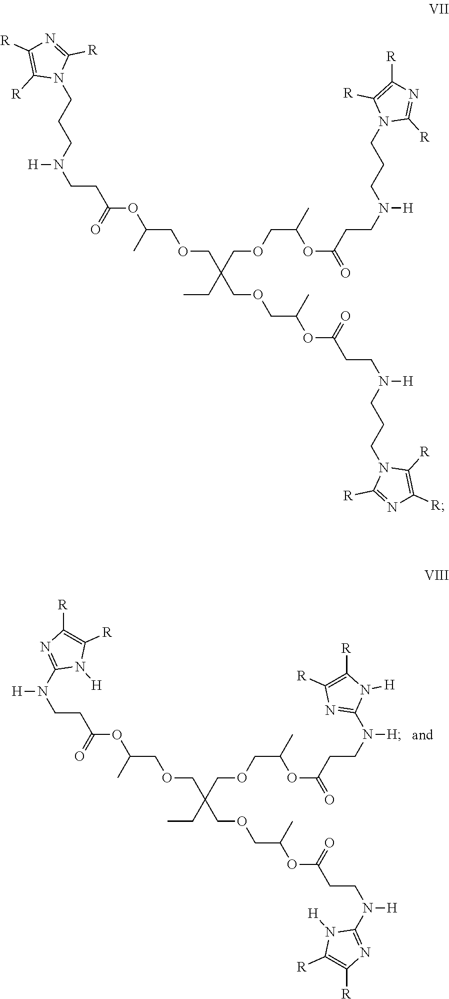 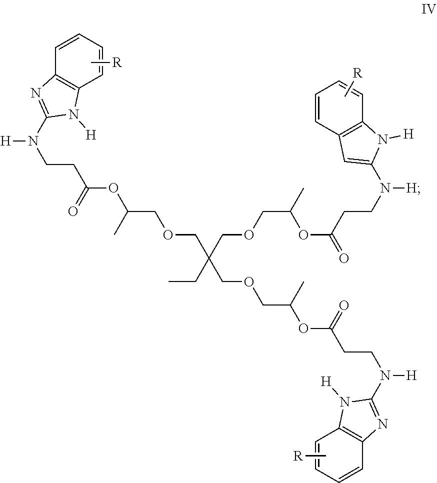
wherein
R=identical and/or nonidentical and ═H, methyl, ethyl, n-propyl, isopropyl, n-butyl, isobutyl, tert-butyl, phenyl and/or mono-, di-, tri-, tetra-, pentasubstituted phenyl, wherein the substituents on the phenyl radical may in turn be identical or nonidentical and may have been selected from the following group:
R_{phenyl}=alkyl (linear and branched), alkoxy, —F, —Cl, —Br, —I, aldehyde, ketone, acyl ester, acyl amide, phosphonic acid derivative and/or sulfonic acid derivative.

12. The wrapping-tape insulating system as claimed in claim 1, wherein the tape accelerator is in the form of a compound whose structure is derived from at least one of the following parent structures X, XI and XII: 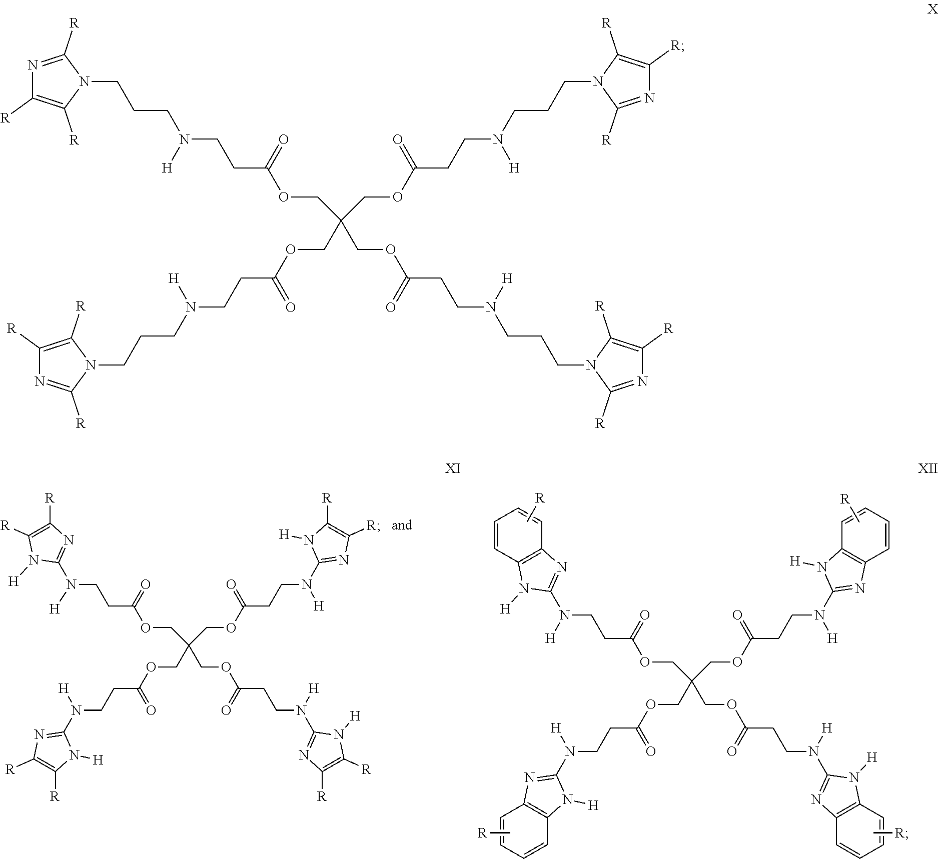
wherein
R=identical and/or nonidentical and ═H, methyl, ethyl, n-propyl, isopropyl, n-butyl, isobutyl, tert-butyl, phenyl and/or mono-, di-, tri-, tetra-, pentasubstituted phenyl, wherein the substituents on the phenyl radical may in turn be identical or nonidentical and may have been selected from the following group:
R_{phenyl}=alkyl (linear and branched), alkoxy, —F, —Cl, —Br, —I, aldehyde, ketone, acyl ester, acyl amide, phosphonic acid derivative and/or sulfonic acid derivative.

13. The wrapping-tape insulating system as claimed in claim 1, wherein the tape accelerator in the form of a compound whose structure is derived from the following parent structure XIII: 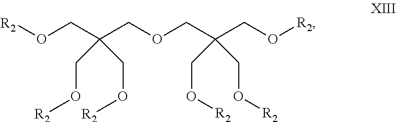
wherein
R₂═H and/or structure XIV 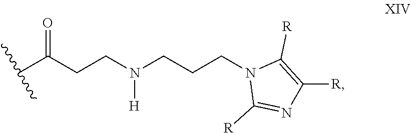
and/or structure XV 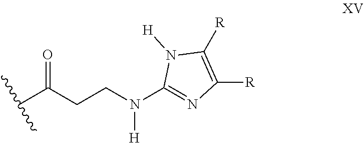
and/or structure XVI 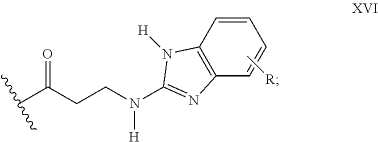
wherein for structure XIII:
R=identical and/or nonidentical and
R═H, methyl, ethyl, n-propyl, isopropyl, n-butyl, isobutyl, tert-butyl, phenyl and/or mono-, di-, tri-, tetra-, pentasubstituted phenyl,
wherein the substituents on the phenyl radical may in turn be identical or nonidentical and may have been selected from the following group:
R_{phenyl}=alkyl (linear and branched), alkoxy, F, Cl, Br, I, aldehyde, ketone, acyl ester, acyl amide, phosphonic acid derivative and/or sulfonic acid derivative
R₂ as indicated in the structures XIV, XV and XVI.

14-15. (canceled)

16. A wrapping-tape insulating system produced by vacuum pressure impregnation, the system comprising:
an insulating tape, the insulating tape comprising
a tape adhesive selected from the group of tape adhesives selected from the group consisting of tricyclomethanedimethanol,trimethylolpropane,hydroxy-functional dendritic polymers,polycaprolactone triols, and polycaprolactone tetrols;
a tape accelerator mixed in the tape adhesive, the tape accelerator selected from the group consisting of 1H-2-aminoimidazoles, 1H-2-(aminoalkyl)imidazoles, and mixtures thereof covalently bonded via the amino function to an acrylate by aza-Michael addition, the acrylate selected from the group consisting of trimethylolpropane triacrylate, trimethylolpropane propoxylate triacrylate, pentaerythritol tetraacrylate,
dipentaerythritol pentaacrylate/dipentaerythritol hexaacrylate and mixtures thereof; and
an anhydride-free resin.

17. The system as claimed in claim 16, wherein the tape accelerator comprises 1,2-dimethylimidazole.

18. The system as claimed in claim 16, wherein the tape adhesive is oxirane group free.

19. The system as claimed in claim 17, wherein the tape adhesive is present in an amount of about 2 wt % of the anhydride-free resin.

20. The system of claim 16, wherein the tape accelerator has a vapor pressure of less than about 10⁻⁴ at a temperature of about 50° C. to about 80° C.

21. The system of claim 16, wherein the tape accelerator has a glass transition temperature of about 140° C. or higher.

22. A wrapping-tape insulating system comprising:
an insulating tape produced by vacuum pressure impregnation, the insulating tape comprising
an oxirane group free tape adhesive;
a tape accelerator mixed in the tape adhesive, the tape accelerator comprising an imidazole covalently bonded via the amino function to an acrylate by aza-Michael addition, the tape accelerator having a vapor pressure of less than about 10⁻³ at a temperature of about 50° C. to about 80° C. and a glass transition temperature of about 140° C. or higher; and
an anhydride-free epoxy resin.

23. The system of claim 22, wherein the imidazole is selected from the group consisting of 1H-2-aminoimidazoles, 1H-2-(aminoalkyl)imidazoles, and mixtures thereof.
